(19) United States
(12) Patent Application Publication
(10) Pub. No.: US 2007/0055196 A1
(43) Pub. Date: Mar. 8, 2007

(54) Implantable shunt system and method

(76) Inventor: William B. Gormley, Weston, PA (US)

Correspondence Address: Loren G. Helmreich;Browning Bushman P.C., Suite 1800, 5718 Westheimer, Houston, TX, 77057, US

(21) Appl. No.: 11/219,434

(22) Filed: Sep. 2, 2005

(51) Int. Cl. A61M 5/00

(52) U.S. Cl. 604/9

(57) ABSTRACT

An implantable shunt system 10 and method include a ventricle catheter 12 for insertion into the brain ventricle, a distal catheter 14 for insertion into a selected terminus in the patient, and a regulating valve 16 for controlling flow through the distal catheter. An implantable pump 24 delivers a liquid thrombolytic solution upstream from the regulating valve, and a check valve 18 prohibits flow through the check valve in the direction toward the brain ventricle. A storage reservoir or chamber 26 is provided for containing the thrombolytic agent.

### FIELD OF THE INVENTION

Many intracranial problems are complicated by the development of intraventricular hemorrhage and secondary hydrocephalus. These problems, regardless of their original etiology, have a common feature of blood in the cerebral ventricular system and secondary hydrocephalus. The present invention provides a more effective treatment strategy for these problems.

### BACKGROUND OF THE INVENTION

Anatomically, the cerebral spinal fluid (CSF) is contained throughout the entire central nervous system. CSF is produced primarily in the lateral, third and fourth ventricles of the brain. It essentially represents an ultrafiltrate of plasma, and in non-disease states does not contain cells or protein. CSF is distributed through out the central nervous system and is held primarily in three different compartments: the ventricles, the subarachnoid space and the lumbar thecal sac. These compartments are in fluid communication and are maintained in a state of homeostasis. The flow of CSF goes from the lateral, to the third and the fourth ventricles, from there to the subarachnoid space surrounding the brain and the spinal cord, and finally to the venous sinuses of the brain where it is re-absorbed.

At any given time, these three primary compartments collectively hold up to 140 cc of CSF. Any increase in this amount rapidly increases the pressure in the system. A total of 500 cc of fluid is produced in 24 hours, and fluid thus has to be completely turned over every 6-7 hours. This occurs, as previously mentioned, by the re-absorption of the fluid by the arachnoid villi located in the venous sinus system of the brain.

Anything which interferes with the normal re-absorption of CSF will rapidly cause an imbalance between production and absorption, and will quickly throw off the homeostasis of the system. When this homeostasis is disturbed, the patient develops hydrocephalus with a rapid increase in intraventricular volume, intraventricular pressure and therefore intracranial pressure. The problem of managing this condition typically-is complicated by the poor clinical condition of these patients. Intracranial problems accompanied by this complication include hypertensive intracerebral hemorrhage with intraventricular extension, subarachnoid hemorrhage with intraventricular hemorrhage, AVM rupture into the ventricular system, tumor hemorrhage into the ventricular system and trauma.

Patients with an intraventricular hemorrhage (IVH) have the normal flow of CSF is mechanically obstructed by the much thicker and often clotted blood in the ventricular system. This clotting quite rapidly leads to an increase in the intraventricular volume and pressure, eventually leading to an increase in intracranial pressure. The treatment for this has been the placement of an externalized ventricular catheter for the decompression of the hydrocephalus and drainage of the intraventricular blood. In some patients, there is only a temporary need for ventricular drainage as the hydrocephalus steaming from the intraventricular hemorrhage is self-limited. Many others, however, will develop long term hydrocephalus, which requires the placement of a ventricular-peritoneal (VP) shunt for its management.

Those patients who develop hydrocephalus and require a ventriculo-peritoneal shunt (VP) placement, have limited absorption of cerebral spinal fluid (CSF) secondary to the scarring caused by the hemorrhage, as well as obstruction in the normal flow of CSF caused by the scarring. When such patients are identified in the acute setting, it is necessary to wait for the blood in the ventricular system to clear before placing a permanent VP shunt. Often it takes between 2-6 weeks of external drainage before the blood in the ventricular system clears. During this time, a VP shunt cannot be placed because the blood in the ventricular system will clog the delicate valve mechanisms in the shunt system. Most of these patients remain in the intensive care unit during the entire period of time required for the blood in the ventricular system to clear enough for the shunt system not to get clogged by the blood products. This extended stay in an intensive care setting represents a two-fold problem. First and most importantly, it is a large risk to the patients who often could otherwise be in a less invasive setting with less potential for infection and other complications, such as a normal ward or a rehabilitation ward. Second, this extended stay represents a large drain on the resources of the facility and the general health system.

In order to mobilize this group of patients sooner and reduce the morbidity and mortality associated with long intensive care settings, and to lower patient cost by reducing their length of stay in the ICU, many attempts at managing the issues associated with intraventricular hemorrhage have been made. Some of the therapies which have been utilized include the use of intraventricular thrombolytics to break down the blood products in the ventricular system. This theoretically would reduce the amount of blood in the ventricular system more promptly and therefore allow for more rapid mobilization of these patients. Although this initially appeared promising, it has had limited application because of the risk of worsening of the intraventricular hemorrhage associated with the thrombolytics. Other attempts have been made to place shunt systems with no valve mechanisms in order to avoid the problem of blood clogging the valve. This has the risk of over drainage of the ventricular system with both short term and long term risks. This strategy places the patient at risk of acute over drainage with its potential catastrophic effects, as well as long term over drainage with alterations in the intracranial pressure (ICP) dynamics.

A fibrinolytic system for use in an IUH application is disclosed in an article by Herbert H. Engelhard entitled “Correct Management of Intraventricular Hermorrage,” Surgical Neurology, Vol. 66, Issue 1, July 2003. A process for reducing cerebrospinal fluid flow obstruction with a clot reducing agent administered intravenously, or by routes including intraventricular and intrathecal, is disclosed in PCT/US03/17271. A cerebrospinal fluid shunt system is disclosed in WO/98/11934.

Another strategy for management of the intraventricular blood has been the use of opened intracranial surgery where the surgeon physically removes the intraventricular blood has been met with generally poor results, and is not felt to be an adequate strategy for the management of this problem. Less catastrophic surgical complications have been encountered with the use of stereotactic techniques to attempt to remove the intraventricular blood. Unfortunately, they also have not had great success in accomplishing the goal of adequately removing the intraventricular blood.

Prior art ventricular drainage systems for the management of hydrocephalus consist of three separate components. The first is the ventricular catheter, the second is the drainage regulating valve, and third is the distal catheter.

The ventricular catheter is simple and consists of a sylastic tube with a few proximal orifices which when located in the ventricle will drain fluid from the ventricle. It rarely is the case of a shunt failure. The distal catheter is likewise a fairly simple component consisting of a second sylastic tube which leads fluid from the distal end of the regulating valve into the terminus which one designates will be the end point for the drained CSF. Normally, this will be the peritoneal cavity but may be the pleural cavity or may even be the circulator system, represented by the atrium of the heart. Although this portion of the shunt fails more commonly than does the ventricular catheter, it seldom fails as a result of mechanical issues of the apparatus, and more often due to physiologic limitations relating to the terminus chosen.

The third component of the shunt system, the regulating valve, overwhelmingly represents the most important segment of the entire system. Most valves are pressure regulating, and control CSF flow rates directly by responding to intraventricular CSF pressure. Most shunt regulating valves are fixed pressure systems that are pre-set by the manufacturer to open and close at established pressures. There have been many recent products that have adjustable pressure regulating valves that can be changed prior to insertion in the patient and even after the valve has been placed. Recently, a new system that functions by controlling CSF drainage through a self-regulating flow valve has been introduced. All of these valve systems, however, have in common extremely small mechanisms positioned within the pathway of CSF drainage. As a result, especially in patients with intraventricular hemorrhage where the CSF has a great deal of blood in it, the valves often fail as they become obstructed with debris. The propensity to become obstructed by blood is common to shunt systems, regardless of the valve mechanism that is utilized.

When a standard VP shunt is placed, it has a catheter going into the ventricle taking CSF via tubing to the valve which is located on the outside of the skull, under the scalp. The valve then connects with a distal catheter that travels in the subcutaneous tissue to the anterior wall of the abdomen. Here, the catheter is placed through the muscular tissue into the peritoneal cavity. The distal end will then float freely in the peritoneum and the CSF will get re-absorbed. The shunt can also be placed in the pleural cavity or even in the circulatory system by way of the right atrium of the heart, although the peritoneum is by far the most commonly utilized terminus.

### SUMMARY OF THE INVENTION

In one embodiment, a cerebral spinal fluid shunt system includes a brain ventricular catheter for insertion into the brain ventricle to drain cerebral spinal fluid from the brain ventricle, and a distal catheter for passing the cerebral spinal fluid to the patient's system. A regulating valve is provided for controlling flow through the distal catheter. A pump delivers a liquid thrombolytic solution to the cerebral spinal fluid upstream from the regulating valve, and a check valve upstream from the regulating valve prohibits flow through the check valve in a direction toward the brain ventricle.

In another embodiment, the cerebral spinal fluid shunt system further includes a solution storage chamber for housing the liquid thrombolytic solution, and a mixing chamber upstream from the regulating valve for mixing the spinal fluid and the liquid thrombolytic solution.

According to one embodiment of the method of the invention, a brain ventricular catheter is inserted into a brain ventricle and a distal catheter extends to a terminus within the patient's system. The catheters are fluidly connected, and a regulating valve provided for controlling the flow through the distal catheter. A pump delivers a liquid thrombolytic solution to the cerebral spinal fluid upstream from the regulating valve, and flow is prohibited through the brain ventricular catheter in a direction toward the brain ventricle.

The new shunting system should be effective in preventing the high rate of shunt failure in the patient population with intraventricular hemorrhage. The shunting system also allows early shunting and the rapid mobilization of patients out of an ICU setting with all.the advantages, both medical and financial, that this would entail.

These and further features and advantages of the present invention will become apparent from the following detailed description, wherein reference is made to the figures in the accompanying drawings.

### BRIEF DESCRIPTION OF THE DRAWINGS

FIG. 1 depicts components of a suitable cerebral spinal fluid shunt system, including a brain venticular catheter, a distal catheter, a regulating valve, pump for delivering a liquid thrombolytic solution to the spinal fluid, and a check valve.

FIG. 2 is a side view of a suitable check valve.

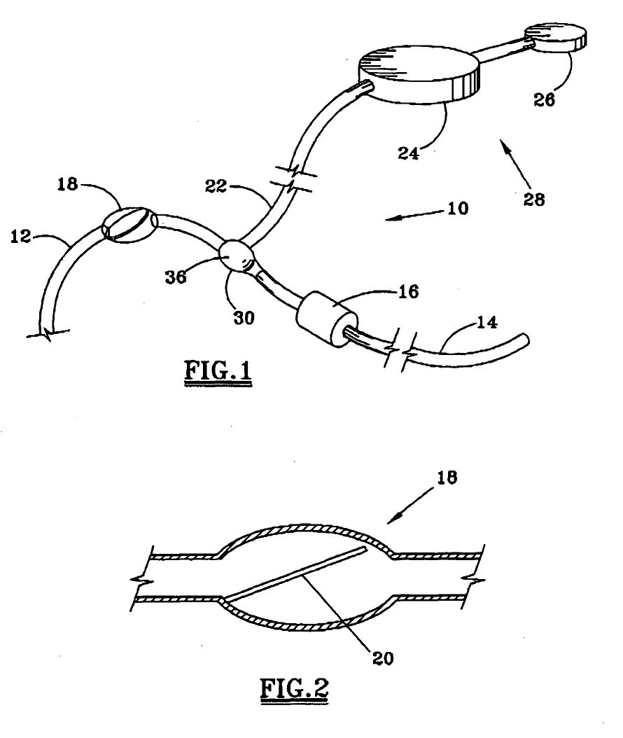

### DESCRIPTION OF THE PREFERRED EMBODIMENTS

The new shunt system 10 utilizes some of the components of existing shunt systems, namely a brain ventricular catheter 12, a distal or downstream catheter 14, and a regulating valve 16, and adds new components in order to overcome the problems particular to the patient population with significant blood in the CSF and its propensity for clogging existing shunt systems. The catheter 12 is positioned for draining fluid from the brain ventricle, and the distal catheter 14 passes the fluid to a selected terminus within the patient's system, such as the peritoneal cavity, the pleural cavity, the pleural cavity, or the blood circulation system. The shunt system function will be independent of the type of regulating valve mechanism utilized and therefore independent of the valve manufacturer. Each catheter preferably includes a sylastic tube.

In order to overcome the problem of the propensity for obstruction of the valve system with blood and debris, the blood is broken down prior to entering the regulating valve. In this way, the likelihood of the valve becoming occluded is dramatically lessened or even eliminated. A thrombolytic agent is introduced into the shunt system before the bloody CSF reaches the regulating valve, preventing the otherwise inevitable obstruction of the valve by the bloody CSF. In order to accomplish this, a number of technical problems have been addressed to maintain the efficacy of the system without creating risk from the system, and specifically from the use of thrombolytics.

The first problem to resolve is the way in which a continuous dose of thrombolytics is infused into the shunt system at a dose rate that is sufficient to create breakdown of bloody CSF. The system used to deliver the thrombolytics has the capacity to deliver the medication through a completely self-contained mechanism. In other words, the entire system may be implantable and does not require any external devises for its use. Second, the system desirably is able to deliver medication to the shunt system for a long enough period of time that external breaching of the system, for refill of medication, for example, should be minimal so as to reduce the risk of infection and the need for dedicated medical assistance. This necessitates that the medication used be very effective in small doses for its thrombolytics effects, and that the reservoir for delivery of the drug be sufficiently large so as to not need frequent refills.

The drug delivery system preferably consists of a drug reservoir fitted with a constant flow output pump, i.e., a pump that dispenses a known quantity of fluid each time the pump is cycled. The pump will be connected to the shunt system via sylastic tubing at a point proximal to and upstream from the regulating valve. In this way, the bloody CSF will mix with the thrombolytics prior to entering the regulating valve. This allows the blood in the CSF to break down prior to reaching the valve, and therefore prevent valve obstruction.

Another aspect to resolve relates to safety. Upstream to the point where the drug reservoir fluidly connects to the system, a one-way valve is incorporated. These valves are “on-off” type valves with little in the way of a mechanism that could get obstructed. The valves do, however, prevent a back flow of CSF mixed with thrombolytics from gaining access into the brain ventricle. In this way, the thrombolytics will only have access to the shunt tubing from the one-way valve and downstream from the one-way valve, including the regulating valve mechanism and the distal tubing. No thrombolytics could enter into the brain ventricle. The risk of subsequent intraventricular hemorrhage induced by thrombolytics should be eliminated.

The components to be added to the traditional shunt system would be three:

1. A one-way or check valve 18 spaced along the tubing 12 coming form the brain ventricle and before reaching the regulating valve 16. The check valve 18 may use a simple flap 20 inside of the tubing (see FIG. 2) that can open only in the direction of forward CSF flow and does not allow retrograde flow. The flap 20 is thus positioned between the brain ventricle end of the ventricular catheter and the regulating valve, and prevents any thrombolytics from gaining access to the ventricular system. The check valve 18 is shown in greater detail in FIG. 2.

2. A reservoir 30 which is intended for the mixing of bloody CSF with thrombolytics. This reservoir will be placed downstream from the one-way valve 18 earlier and upstream of the regulating valve 16 of the shunt system. This reservoir will have two inlets, one to accept the bloody CSF coming from the ventricular system, and the second to accept the thrombolytic agent coming from the pump. This reservoir will allow an area for the mixing of the bloody CSF and the thrombolytics that will break down of blood products prior to them reaching the valve. The mixing reservoir 30 preferably has a cross-section greater than that of the upstream and downstream catheters, and preferably has a volume of from about 1 to about 5 cc. In some applications, the mixing reservoir or chamber may not be used, although the junction of the ventricular catheter and the liquid thrombolytic solution from the pump preferably include a small chamber where mixing occurs before the mixed fluid is passed to the regulating valve.

3. A drug delivery system 28 with an implantable pump 24. This may be simple pump with a set rate of drug delivery. The device will be fully implantable in the subcutaneous tissue where it will be accessible for refilling with medication. Reservoir 26 contains the thrombolytic agent of sufficient size (10-30 cc volume) so as to make the need for refill no more than once weekly. The pump 24 may be connected to the shunt system via a sylastic tube 22 and its point of connection to the system will be one of the inlet ports of the reservoir described above. Since the intraventricular blood dissolves over the course of 2-6 weeks, the long-term function of the pump only needs to be for a period of 6 weeks. At this point it can be removed through a simple operation or it can be left in place indefinitely.

The ventricular catheter 12 may be placed into the brain ventricle through a burr hole in the skull. The one-way valve 18 would connect to it and lie on the external surface of the skull, under the scalp. It would connect with the three-way connector 36 which contains the reservoir 30 and then to the regulating valve 16, which will also be on the surface of the skull, below the scalp. The distal catheter 14 may then be placed under the subcutaneous tissue and extend to a selected terminus within the patient. The implantable drug delivery pump 24 and reservoir 26 may be placed under the subcutaneous tissue of the anterior chest wall, and then its connecting tubing connected to the three-way valve under the scalp. The pump 24 and reservoir 26 may be made as a single unit, and may be external to or implanted under the patient's skin.

The system may be a completely implantable device that can be utilized while the patient still has significant amounts of blood in the CSF. This will allow for early mobilization of patients without having the complication of shunt malfunction due to obstruction of the regulating valve system by bloody CSF. The benefits will be dramatic, both for the patient by reducing morbidity and mortality associated with infections and other complications associated with externalized ventricular catheters, and for the health care system by reducing costs associated with increased length of stay in the intensive care settings.

The term “check valve” as used herein is broadly referring to any type of valve for allowing fluid flow in one direction and substantially prohibiting fluid flow in an opposite direction. A check valve with a flapper member is preferred, but other types of valves may serve the same purpose. The term “regulating valve” as used herein is intended to refer to any valve for controlling flow through the distal catheter. Various types of regulating valves have been used for selectively controlling the passage of cerebral spinal fluid to a selected terminus within the patient, and may be used with the shunt system of the present invention. Similarly, various types of pumps have been devised for delivering a liquid thrombolytic solution to a delivery location upstream from the regulating valve. A constant flow output pump which dispenses a given quantity of fluid each time the pump is cycled is preferred for many applications.

Although specific embodiments of the invention have been described herein in some detail, this has been done solely for the purposes of explaining the various aspects of the invention, and is not intended to limit the scope of the invention as defined in the claims which follow. Those skilled in the art will understand that the embodiment shown and described is exemplary, and various other substitutions, alterations and modifications, including but not limited to those design alternatives specifically discussed herein, may be made in the practice of the invention without departing from its scope.

What is claimed is:

1. A cerebral spinal fluid shunt system, comprising:
a brain ventricle catheter for draining cerebral spinal fluid from the brain ventricle;
a distal catheter for passing the cerebral spinal fluid to a selected terminus within a patient;
a regulating valve for controlling flow through the distal catheter;
a pump for delivering a liquid thrombolytic solution to the cerebral spinal fluid at a delivery location upstream from the regulating valve; and
a check valve upstream from both the regulating valve and the delivery location for prohibiting flow through the check valve in a direction toward the brain ventricle.

2. A system as defined in claim 1, further comprising:
a solution storage chamber for housing a liquid thrombolytic solution; and
the storage chamber and the pump are implanted under the tissue of the anterior chest wall of the patient.

3. A system as defined in claim 1, wherein the check valve includes a flapper member.

4. A system as defined in claim 1, further comprising:
a connector for mixing cerebral spinal fluid with the liquid thrombolytic solution at the delivery location upstream from the regulating valve.

5. A system as defined in claim 1, further comprising:
a mixing chamber upstream from the regulating valve for mixing the spinal fluid and the liquid thrombolytic solution.

6. A system as defined in claim 1, further comprising:
a solution storage chamber for supplying a liquid thrombolytic solution to the pump.

7. A system as defined in claim 6, wherein the storage chamber has a volume of from 10 to 30 cc.

8. A system as defined in claim 1, wherein the pump has a constant fluid flow output each time the pump is cycled.

9. A system as defined in claim 1, wherein each of the brain ventricular catheter and the distal catheter includes a sylastic tube.

10. A system as defined in claim 1, wherein the check valve and the regulating valve are positioned under a patient's scalp.

11. A cerebral spinal fluid shunt system, comprising:
a brain ventricle catheter for insertion into the brain ventricle to drain cerebral spinal fluid from the brain ventricle;
a distal catheter for passing the cerebral spinal fluid to a selected location within a patient;
a regulating valve for controlling flow through the distal catheter;
a solution storage chamber for housing a liquid thrombolytic solution;
a pump for delivering the liquid thrombolytic solution to the cerebral spinal fluid at a delivery location upstream from the regulating valve;
a mixing chamber upstream from the regulating valve for mixing the spinal fluid and the liquid thrombolytic solution; and
a check valve upstream from both the regulating valve and the delivery location for prohibiting flow through the check valve in a direction toward the brain ventricle.

12. A system as defined in claim 11, wherein the check valve includes a flapper member.

13. A system as defined in claim 11, wherein the storage chamber has a volume of from 10 to 30 cc.

14. A system as defined in claim 11, wherein each of the brain ventricular catheter and the distal catheter includes a sylastic tube.

15. A system as defined in claim 11, wherein the check valve and the regulating valve are positioned under a patient's scalp.

16. A method for draining cerebral spinal fluid, comprising:
inserting a brain ventricle catheter to drain cerebral spinal fluid from the brain ventricle;
inserting a distal catheter to pass the cerebral spinal fluid to a selected terminus within a patient;
fluidly connecting the ventricle catheter and the distal catheter;
providing a regulating valve for controlling flow through the distal catheter;
providing a pump for delivering a liquid thrombolytic solution to the cerebral spinal fluid at a location upstream from the regulating valve; and
prohibiting flow through the brain ventricle catheter in a direction toward the brain ventricle.

17. A method as defined in claim 16, further comprising:
providing a solution storage chamber for housing the liquid thrombolytic solution; and
implanting the storage chamber and the pump under the tissue of the anterior chest wall of the patient.

18. A method as defined in claim 16, further comprising:
mixing cerebral spinal fluid with the liquid thrombolytic solution upstream from the regulating valve.

19. A method as defined in claim 16, wherein the storage chamber has a volume of from 10 to 30 cc.

20. A method as defined in claim 16, wherein the pump has a constant flow output each time the pump is cycled.
